(19) United States
(12) Patent Application Publication
(10) Pub. No.: US 2008/0131536 A1
(43) Pub. Date: Jun. 5, 2008

(54) Drug for treatment of complications of diabetes mellitus

(76) Inventor: Ahmed Mohamed Ali Massoud, Heliopolis (EG)

Correspondence Address: PYLE & PIONTEK;ATTN: THOMAS R. VIGIL, 221 N LASALLE STREET , ROOM 2036, ROOM 2036, CHICAGO, IL, 60601, US

(21) Appl. No.: 11/986,748

(22) Filed: Nov. 26, 2007

(30) Foreign Application Priority Data
Nov. 26, 2006 (EG) 609/2006

(51) Int. Cl. A61K 9/48; A61K 36/236; A61P 3/10

(52) U.S. Cl. 424/757; 424/451

(57) ABSTRACT

The method is used to treat diabetic complications such as neuropathy, peripheral neuritis, cataract and retinopathy have shown improvement with flavonoids present in licorice. The improvement was found to be due to the prevention of transformation of glucose to sorbitol by inhibiting aldose reductase enzyme, thus avoiding the accumulation of sorbitol in several tissues including lens, retina, kidney, blood vessels, peripheral nerves and striated muscles. Additionally, licorice prepared by fermentation is rich in amylase which improves diabetic complications due to increase of cellular enzymatic activity. This drug was prepared by fermentation of licorice followed by getting rid of glycerizine using hydrochloric acid and not by boiling for fear of destruction of enzymes and flavonoids.

### FIELD OF THE INVENTION

The present invention relates to a method for preparing a medication for treating diabetes mellitus

### BRIEF DESCRIPTION OF THE PRIOR ART

The total number of diabetics allover the world is more than 300 millions (5-6% of world population). There are two types of diabetes mellitus:
1—Type one diabetes mellitus which is also called (insulin dependent diabetes mellitus). It represents about 10% of total diabetic cases and is treated by insulin only.
2—Type two diabetes mellitus which is also called (non insulin dependent diabetes mellitus), or adult type. It represents about 90% of total diabetic cases. It is treated by oral anti-diabetic drugs which aim at reducing blood sugar level. They can be classified into 3 main groups
A—Sulphonyl ureas.
B—Metformin.
C—Insulin sensitizers which activate insulin receptors in body cells. This group of drugs is called thiazolidine diones which includes rosiglitazone as avandia, pioglitazone as glustin.

### Disadvantages in Previous Art

The present problem for diabetic patients is the persistence of complications which increase by time inspite of treatment, as all drugs used today aim at lowering the blood sugar level through:
A—Injection of insulin.
B—Stimulation of insulin secretion by beta cells of pancreas (action of sulphonyly ureas).
C—Lowering blood glucose through direct stimulation of glycolysis, slowing of absorption of glucose in intestine and reduction of gluconeogenesis in liver (action of biguanides).
D—Insulin sensitizer (action of thiazolidine diones which include pioglitazone as glustin and rosiglitazone as avandia). But the main problem of diabetes—besides hyperglycemia—is the severe and multiple complications of the disease that increase and become more severe by time even in case of success in blood sugar control. The most important complications of diabetes are:
   1—Cataract and retinopathy that may cause diminution of vision.
   2—Nephropathy that may end in renal failure. Diabetes mellitus is one of the most important causes of renal failure.
   3—Peripheral neuritis which may cause pain, numbness and loss of sensation. This is a serious complication as it may end in foot gangrene in case of unnoticed trauma because of the present peripheral arteritis and poor blood supply of foot which are common in diabetes mellitus.
   4—Peripheral arteritis.
   5—Cerebral vascular disease.
   6—Hypertension.
   7—Coronary insufficiency.
   8—Rise in serum uric acid.
   9—Atherosclerosis and dyslipidemia.
   10—Hyper coagulability.

All complications of diabetes mellitus persist and become more intensified by time inspite of the regular follow up and treatment of hyperglycemia, all available treatment for these complications are only symptomatic. This is a big problem which necessitates searching for the etiology of these complications and hence the etiological treatment, other than the symptomatic treatment used nowadays.

### Advantages of the New Drug

The drug or medication of the present invention aims at treatment of complications of diabetes mellitus which are very common. They persist and become more intensified by time in spite of control of blood sugar levels. These complications represent the most serious problem of diabetes mellitus. To search for a drug that can prevent these complications, we had to study the main role of enzymes in metabolic reactions allover the body because

There is a strict relationship between enzymatic deficiency and the metabolic disturbances which cause all diabetic complications.

This was confirmed by the following facts:

### First:

All metabolic activities in the body are carried out in the form of chain of enzymatic reactions. Deficiency of specific metabolic enzymes causes abnormal metabolism (6-22). Researches concerning diabetic complications showed that these complications start, by disturbance of glucose metabolism inside the cell and tissues of diabetic patient. Normally glucose catabolism is the major source energy for cellular processes. In diabetic patient, disturbance of glucose metabolism in cells will be accompanied and complicated by the following:
A—Liver glycogen is depleted (5).
B—Protein becomes the only important source of energy. This causes great increase of protein catabolism, decrease of protein synthesis in muscles which end in muscle weakness.
C—Great increase of fat catabolism which causes several complications caused by dyslipidemia. Thus, the main problem that causes all these complications is the disturbance of glucose metabolism in cells which is carried out—as mentioned—in the form of enzymatic reactions—by specific metabolic enzymes.

### Second:

Recent researches in hepatology showed that complications of diabetes mellitus are associated with mitochondrial dysfunction in liver cells. Mitochondria in the liver cells are the site of energy production which enables these cells to carry out their normal metabolic functions. Mitochondria also represent the cross roads for the three major metabolic pathways, carbohydrate, lipids and protein. Thus, they render the liver capable of directing one metabolic pathway in favor of the others in order to attain the metabolic balance required. The relationship between the degree of mitochondrial disturbance and the degree of diabetic complications is of great importance (14, 15, 16).

### Third:

Recently it was discovered that the role of what is called (digestive enzymes) is not digestion only, but they are absorbed in the intestine, they are carried to the liver where they have an important role in metabolic reactions in the liver and all body tissues (6, 8, 9, 17, 18, 19)

It was proved also that pancreatic amylase—besides its role in digestion—is absorbed in the intestine and reaches the liver. This circulation was called entero-hepatic circulation and entero-pancreatic circulation (6)

Besides, researches showed that deficiency in food enzymes leads to hypertrophy of pancreas and salivary glands to compensate for digestive enzymes, and this hypertrophy can be relieved by oral supply of sufficient amounts of these enzymes. If deficiency of these enzymes in diet for a longer time it may cause deficiency of metabolic activities all over the body (6)

Accordingly, we searched for a drug that can treat the presenting complications of diabetes mellitus gradually and prevent further complications and deterioration by supplying the patient with sufficient amounts of enzymes which are necessary for the normal enzymatic activity to regain the normal metabolism allover the body. As for the enzymes to be used we gave the priority to amylase enzyme, as it is the enzyme concerned with carbohydrate metabolism. Also liver cells identify this enzyme and receive it from the blood through the specific receptors called mannose receptors which are found on liver cell surfaces (13)

Besides, amylase enzyme has high affinity to liver glycogen (1). All above mentioned facts confirm the important role of amylase in carbohydrate metabolism and in liver metabolism which affects all body functions.

Also we searched for a common plant which is rich in enzymes. We tried to avoid synthetic drugs or strange substances which may not be easily absorbed. It is well known now that nutrients are absorbed through the intestinal enterocytes by active transport which moves the nutrients against chemical and electrical gradients, a process called active transport (24). Absorption of enzymes is affected by several factors; a very important factor is the mixture containing the enzymes (6).

Licorice was chosen as the rich source of amylase enzyme for the following reasons:

1—it is a natural source of plant origin, and is used in Egypt as a popular drink after being fermented since hundreds or even thousands of years. To get a big amount of amylase in the prepared licorice, our preparation was concentrated 80 times that of the preparation prepared in the traditional way
2—it was discovered that licorice is very rich also in flavonoids (more than 50 substances) (1, 29) which are very important antioxidants and help to regain normal metabolism and treat diabetic complications. Recent researches confirmed that flavonoids inhibit aldose reductase enzyme, thus preventing the conversion of excess glucose to sorbitol. The accumulation of sorbitol (in lens, retina, renal tissue, blood vessels, striated muscles, peripheral nerves, etc.) Is the cause of complications of diabetes mellitus (2, 3, 4, 28). Some researchers stated that inhibitors of aldose reductase would be the potent agents for the prevention of diabetic complications (27). Others stated that flavonoids are the most promising compound for the prevention and treatment of diabetic complications (26). Many experts showed that flavonoids in licorice had important therapeutic effects for the following complications of diabetes mellitus:
A—Cataract (1).
B—Peripheral neuritis (1).
C—Nephropathy (1).
D—Retinopathy (1).
   It is worth mentioning in that respect that there is a close relation, that has not been clarified before, between retinopathy and nephropathy. These two complications occur simultaneously after about 10-15 years from the beginning of the disease and gradually increase in intensity at almost the same rate. The appearance of one of them heralds the appearance of the other and nearly with the same intensity. Experimental work has proved that flavonoids present in licorice, as already mentioned, cure these two complications together by preventing the accumulation of sorbitol in renal and retinal tissues as well as in other tissues which are responsible for some other serious diabetic complications.
E—Prevention of intra vascular clotting through anti-platelet aggregation (30, 31).
F—Prevention of atherosclerosis: by several flavonoids in licorice, the most potent of which are polyphenolic flavonoids (32, 33, 34, 35).
G—Protection of hypercholesterolemia by preventing the peroxidation and damage of low density lipoprotein (32, 35). Thus preventing hypercholesterolemia which occurs as a result of destruction of low density lipoproteins. These lipoproteins are being destroyed by microphages due to their denaturation and peroxidation
H—Reduction of plasma lipids levels (32)
I—Decrease systolic blood pressure (32)
   J—Improving liver metabolism: this was manifested by inhibition of cytochrome oxidase p450 enzyme which becomes higher in diabetics than in normal person indicating the presence of impaired metabolism in the liver of diabetic patients (7,10). Flavonoids have been shown to be potent inhibitors of cytochrome enzyme p450 (7). It is worth mentioning that the improved metabolism in liver cells in diabetes by the use of flavonoids explains the success of these flavonoids in treating many important diabetic complications due to disturbed liver metabolism such as dislipidemia, hypercogulabilty atherosclerosis, coronary artery disease and others. Recent researches by hepatologists confirmed that these complications occur in diabetics with the beginning of appearance of disturbed liver metabolism starting as non-alcoholic steatohepatitis. Intensity of these complications increases with deterioration of the mitochondrial function in hepatocytes (12,14).

### How do Flavonoids Prevent Diabetic Complications in Different Body Tissues?

Many researches have recently proved that flavonoids help in treatment and prevention of diabetic complications by preventing the accumulation of sorbitol in different body tissues. This is achieved by suppression of aldose reductase enzyme which initiates the first step in polyol pathway which occurs as a result of elevation of glucose concentration in tissues above normal levels which ends up in transformation of glucose into sorbitol (25,26,27,28).

### How do Flavonoids Regain Normal Vitality and Functions of Hepatocytes?

Flavonoids act as antioxidants. They regain normal enzymatic activity within hepatocytes by preventing accumulation of reactive oxygen species (ROS) that result as by-product of metabolic activity within the mitochondria (36). Inside theses mitochondria oxidative reduction reactions take place aiming at production of energy in form of ATP by the help of some important enzymes and coenzymes such as ATP synthase, cytochrome c oxidase, nicotinamide adenine dinucleotide (NAD) and flavine adenine dinucleotide (FAD). During these continuous enzymatic reactions small amount of reactive oxygen species (ROS) results which threatens to destroy cellular constituents. Some of NAD and FAD deal with these small amounts of ROS to protect cellular contents from destruction (16,23). The rest of NAD and FAD play their role in the process of normal metabolism and energy production inside cells. In diabetes mellitus, abnormal metabolism will result in accumulation of big amounts of ROS needing big amounts of NAD and FAD to deal with, with consequent deterioration in functions of NAD and FAD and their role in energy production and normal metabolism inside liver cells due to this abnormal pathway and abnormal consumption. Flavonoids deal and destroy excess ROS, thus sparing NAD and FAD to perform their normal activity in energy production and normal metabolism inside liver cells so liver regains its normal vitality and functions (36).

### REFERENCES

1—Carol A. Newall, Linda A. Anderson and David J. (1996): Herbal medicines, a guide for health care professionals. London, the pharmaceutical press.
2—Aida K. E. et al. (1990): Isoliquiritigenin: A New Aldose Reductase Inhibitor From Glycyrrhizae Radix Planta Med, 56: 254-8.
3—Yun-Ping Z., Jia-Qing Z. (1989): Oral baicalin and liquid extract of licorice reduce sorbitol levels in red blood cells of diabetic rats. Chin Med J, 102: 203-6.
4—Inque H. et al. (1987): Pharmacological activities of glycerrhetinic acid derivatives: analgesic and anti-type allergic effects. Chem Pharm Bull, 35: 3888-93.
5—Ganong W. (1993): Review of medical physiology, 16^{th} (edit) prentice-hall international inc. (pub), (19); 310.
6—Lopez D. A., Williams R, M and Miehike S. (1994): Enzymes the fountain of life, 1st (edit), the neville press inc. (pub) Munchen, 34-37.
7—Hamdy A. M. (2000): Biochemical study on the effects of some Egyptian herbs in alloxan—induced diabetic rats, toxicology 174, 131-139.
8—Rosenblum J. L., Raab B. K. and Alpers D. H. (1982): Hepatobiliary and pancreatic clearance of circulating pancreatic amylase Am J Physoil., July; 243 (1):21-7.
9—Leibow C. and Rothman S. S. (1975): Enteropancreatic circulation of digestive enzymes. Science 8; 189 (4201): 472-4.
10—Dessayre D., Berson A., Fromenty B. and Mansouri A. (2001): Mitochondrial Dysfunction In Hepatic Steatosis In Leuschner U, James O And Dancygier H (Edit) Falk Symposium 121 Steatohepatitis (Nash And Ash), Kluwer Academic Publishers (Pub), London; 80-100.
11—Koyqma I. K. and Hokari S. (2001): Expression of alpha amylase gene in rat liver: liver-specific amylase has a high affinity to glycogen. Electrophoresis; 22 (1): 12-7.
12—Lonardo A., Loria P. and Carulli M. (2001): Insulin Resistance In Non—Alcoholic Fatty Liver Disease: A Clinical Perspective In Leuschner U, James O And Dancygier H (Edit) Falk Symposium 121 Steatohepatitis (Nash And Ash), Kluwer Academic Publishers (Pub), London; 104-113.
13—Niesen T. E., Alpres D. H., Rosenblum J. L. (1984): Metabolism of glycosylated human salivary amylase in vivo plasma clearance by rat hepatic endothelial cells and in vitro receptor mediated pinocytosis by rat macrophages. J Leukoc Boil; 36 (3): 307-20.
14—Sanyal A J, Campbell-Sargent C, Mirshahi F, Rizzo W B Contos M J and Clore J (2001): The intrahepatic and extrahepatic metabolic abnormalities associated with non-alcoholic steatohepatitis. In leuschner U, James O and Dancygier H (edit) Falk symposium 121 Steatohepatitis (NASH and ASH), Kluwer Academic Publishers (Pub) London; 172-81.
15—Schalm S. W. and Von Hoek B. (2001): Treatment of non-alcoholic steatohepatitis role of ursodeoxycholic acid. In leuschner U, James O and Dancygier H (edit) Falk symposium 121 Steatohepatitis (NASH and ASH) Kluwer Academic Publishers (Pub), London; 185-187.
16—Fromenty B., Degoul F., Demeilliers C., Sutton A., Mansouri A. and Pessayre D. (2001): Alcohol and mitochondrial function in leuschner U, James O and Dancygier H (edit) Falk Symposium 121 Steatohepatitis (NASH and ASH) Kluwer Academic Publishers (Pub) London; 257-275.
17—Matthews D. M. (1992): Protein absorption. Wiley-Liss, New York.
18—Siamak A. A. (1997): The oligopeptide transporter (pept-1-) in human intestine: biology and function. Gastroenterology; 113:332-340.
19—Curr Drug Metab (2004); 5 (1): 85-94
20—Sterling M. R. (1999): Can cirrhosis be prevented?, Issue of Nutrition Science News Jan.
21—Tom Brody (2001): Anti oxidants. in nutritional biochemistry, 2^{nd} edition. Academic Press.
22—Dufour J. F. (2001): Anti oxidants in the treatment of non alcoholic steatohepatitis. In Leuschner U, James O and Dancygier H (edit) Falk symposium 121 Steatohepatitis (NASH and ASH) Kluwer Academic Publishers (Pub) London; 197-202.
23—Balz F. (1994): Reactive oxygen species and antioxidant vitamins. American journal of medicine, vol. 97, page 3a-7s, by Excerpta Medica.
24—Phillip P. T. (1996): Malabsorption. in Claude Bennett and Fred Plum, Cecil Textbook of Medicine, 20^{th} edition. W.B. Saunders company.
25—Zhou Y., Zhang J. (1990): Effects of baicalin and liquid extract of licorice on sorbitol level in red blood cells of diabetic rats-Zhongguo Zhong Yao Zazhi.; 15 (7): 433-5, 448.
26—Lim S. S., Jung S. H., Ji J., Shin K. H., Keum S. R. (2001): Synthesis of flavonoids and their effects on aldose reductase and sorbitol accumulation in streptozotocin-induced diabetic at tissues. Pharmacol.; 53(5): 653-68.
27—Jung S. H., Lee Y. S., Shim S. H. (2005): Inhibitory effects of Ganoderma applanatum on rat lens aldose reductase and sorbitol accumulation in streptozotocin-induced diabetic rat tissues. Phytother Res.; 19(6): 477-80.
28—Cameron N. E., Cotter M. A., Basso M. (1997): Comparison of the effects of inhibitors of aldose reductase and sorbitol dehydrogenase on neurovascular function, nerve conduction and tissue polyol pathway metabolites in streptozotocin-diabetic rats. Diabetologia.; 40(3): 271-81.
29—Kitagawa L., Chen W. Z., Taniyama T., Harada E., Hori K., Kobayashi M., Ren J. (1998): Quantitative determination of constituents in various licorice roots by means of high performance liquid chromatography. Yakugaku Zasshi.; 118(11): 519-28.
30—Zhan C., Yang J. (2006): Protective effects of isoliquiritigenin in transient middle cerebral artery occlusion-induced focal cerebral ischemia in rats. Pharmacol Res.; 53 (3): 303-9.
31—Tawata M., Aida K., Noguchi T., Ozaki Y., Kume S., Sasaki H., Chin M., Onaya T. (1992): Anti-platelet action of isoliquiritigenin an aldose reductase inhibitor in licorice. Eur J Pharmacol, 25; 212 (1): 87-92.
32—Fuhrman B., Volkova N., Kaplan M., Presser D., Attias J., Hayek T., Aviram M. (2002): Anti atherosclerotic effects of licorice extract supplementation on hypercholesterolemic patients. Nutrition.; 18(3): 268-73.
33—Aviram M., Fuhrman B. (1998): Polyphenolic flavonoids inhibit microphage-mediated oxidation of LDL and attenuate atherogenesis. Atherosclerosis. 137 Suppl: 45-50.
34—Belinky P. A., Aviram M., Fuhrman B., Rosenblat M., Vaya J. (1998): The antioxidative effects of the isoflavan glabridin on endogenous constituents of LDL during its oxidation atherosclerosis.; 137(1): 49-61.
35—Fuhrman B., Buch S., Vaya J., Belinky P. A., Coleman R., Hayek T., Avirm M. (1997): Licorice extract and its major polyphenol glabridin protect low-density lipoprotein against lipid peroxidation: in vitro and ex vivo studies in humans and in atherosclerotic apolipoprotein E-deficient mice. Am 3 Clin Nutr.; 66(2): 267-75 36 Haraguchi H., Yoshida N., Ishikawa H. (2000): Protection of mitochondrial functions againts oxidative stresses by isoflavans from glycyrhiza glabra. J Pharm Pharmacol.; 52(2) 219-23.

### DETAILED DESCRIPTION

This drug is characterized by the following:
a—Mode of preparation of licorice in this drug differs from that of all preparations of licorice used for therapeutic purposes which deal with licorice root. This drug is prepared by fermentation of licorice for 6 hours (using water and sodium bicarbonate). The aim of fermentation is the production of large amounts of enzymes that treat with glucose. Root of licorice contains 30% starch, 2% glucose and 5% sucrose
b—Mode of exclusion of glycerizine in this drug differs from that used for getting deglycerizinized licorice (dgl) by boiling. here glycerizine is excluded by using diluted hydrochloric acid to protect the prepared enzymes from the effect of hyperthermia, as these enzymes may be damaged if exposed to temperature above 60 c.

### The Following Materials are Used for Preparation of the Drug:

1 Licorice root.
2—HCl (4 molar).
3—Sodium carbonate.

### Preparation of the Drug in the Form of Syrup:

1—250 gram of licorice root & 0.2 gram of sodium carbonate are added to 500 c.c. of warm water. the mixture is left for 6 hours.
2—750 ml of warm water is added to the mixture and left for 4 hours.
3—Diluted HCl is added then the mixture is stirred and filtered.
4—The filtrate is used as a drug. the dose is 5 ml (one tea spoonful) TDS.

### Preparation of the Drug in the Form of Capsules:

a—The drug is prepared in the form of soft gelatinous capsules according to the following steps:
   1—The solution is prepared according to the above mentioned steps.
   2—Reduction of water content is done by rotary suction pump.
   3—The produced dough is packed in soft gelatinous capsules. Each capsule contains a dose equivalent to 5 ml of syrup form.
b—The drug is prepared in the form of hard capsules as follows:
   1—The solution is prepared according to the above mentioned steps.
   2—Dryness is done by lyophilization.
   3—Each capsule contains a dose equivalent to 5 ml syrup form.

The effect of drug on some complications
of diabetes in 62 patients

| | | No. Of | |
|---|---|---|---|
| | Complication | cases | Effect of the drug |
| 1 | somnolence, the desire to | 23 | complete resolution within |
| | relax or sleep for long hours | | 3-4 wks in all cases. |
| 2 | fragility and easy | 24 | dramatic improvement in |
| | fatigability | | all cases within 3-4 wks. |
| 3 | shoulder pain and limitation | 13 | complete resolution |
| | of shoulder movement (up to | | within 3-4 wks. |
| | frozen shoulder | | |
| 4 | joint and bone aches in the | 17 | pains relieved in less |
| | hands, limbs, chest and toes, | | than 4 wks. in all cases |
| | severe enough to keep | | |
| | patients awake all night. | | |
| 5 | boils and skin infections | 14 | complete disappearance |
| | (recurrent). | | within 3-4 wks. |
| 6 | slow healing of wounds | 5 | resolved in all cases |
| | | | within 2-3 months |
| 7 | brittle finger nails | 7 | resumed their natural |
| | | | fitness within 2-3 months |
| 8 | hair falling off (heavily | 7 | hair regained its vivid- |
| | | | ness and stopped falling |
| | | | off within 6-8 wks. |
| 9 | weak sensation in both feet | 13 | dramatic improvement in |
| | | | all cases within 3-4 wks. |
| | | | (some cases even resumed |
| | | | their “normal” |
| | | | sensation. |
| 10 | tingling and numbness in the | 28 | resolved “completely” |
| | limbs and the trunk. | | in 7 cases and improved |
| | | | remarkably in the |
| | | | remaining cases within |
| | | | 3-4 wks. |
| 11 | a sense of abnormal warmth | 6 | resolved completely in |
| | in the sole of the feet. | | all cases within 3-4 wks. |
| 12 | cramps of the limbs (severe | 17 | resolved completely in |
| | enough to keep patients a- | | all cases within 3-4 wks. |
| | wake by night in some cases. | | |
| 13 | systemic primary | 32 | a-improved response to |
| | hypertension | | antihypertensive treat- |
| | | | ment in 5 cases (blood |
| | | | pressure range was 160- |
| | | | 170/100-105- inspite of |
| | | | antihypertensive treat- |
| | | | ment) the pressure dropped |
| | | | to normal range with the |
| | | | use of the drug. |
| | | | b-significant reduction |
| | | | of the dose of antihyper- |
| | | | tensive drugs in 8 cases |
| | | | c- in one case: (case no. |
| | | | 43) the blood pressure |
| | | | dropped to normal range |
| | | | (120/80) and antihyper- |
| | | | tensive were discontinued |
| | | | (the patient was resistant |
| | | | to antihypertensive before |
| | | | the use of the drug and |
| | | | the blood pressure was |
| | | | around 170/95 mmHg.) |
| | | | d- in the rest of cases, |
| | | | the blood pressure was |
| | | | almost normalized with |
| | | | the use of the usual dose |
| | | | of antihypertensive drugs |
| | | | along with the use of |
| | | | the drug. |
| 14 | weight loss (and even | 12 | patient resumed normal |
| | emaciation in some cases) | | weight within 3-4 wks. |
| | inspite of normal appetite | | |
| | and normal food intake | | |
| 15 | abnormal hyperphagia | 4 | patients regained normal |
| | | | appetite (with the loss of |
| | | | the sense of continuous |
| | | | hunger) within 2-3 wks |
| 16 | cataract | 1 | disappeared after one |
| | | | year of treatment (the |
| | | | diagnosis was confirmed |
| | | | by slit-lamp examination). |
| 17 | haziness and blurring of | 5 | completely resolved in |
| | vision as a result of weak | | all cases within 3-4 wks. |
| | ocular muscles. | | |
| 18 | weak body resistance | 6 | all cases significantly |
| | presenting as upper respi- | | improved within 4-6 month |
| | ratory tract infections | | as interpreted by the |
| | recurring at remarkably | | significant reduction of |
| | short intervals and requir- | | both the number and |
| | ing antibiotic treatment | | severity of attacks |
| | | | within that period. |
| 19 | amenorrhoea (in absence of | 1 | menstrual cycles recurred |
| | pregnancy) in a 26 years | | in a regular manner after |
| | old patient. | | two months of the use of |
| | | | the drug (after being |
| | | | lost for over a year). |
| | | | the patient got pregnant |
| | | | after 5 months of treatment. |
| 20 | bowel disturbances and | 5 | Resolved completely in |
| | severe constipation. | | all cases within 3-4 wks. |
| 21 | diabetic foot with | 1 | the condition improved |
| | ulceration and impending | | dramatically with the use |
| | gangrene | | of the drug and the deci- |
| | | | sion of below-knee ampu- |
| | | | tation was ruled out |
| | | | within 3-4 wks. |
| 22 | fatty liver | 25 | only one case was |
| | | | followed up by repeated |
| | | | sonography. fatty liver |
| | | | disappeared after a few |
| | | | months and did not recur |
| | | | in the sonogram taken one |
| | | | year later |

Comments on a Few Cases Whose Response with the Use of the Drug was Dramatic:
a—Case 3:

a 20 years old patient could hardly tolerate walking for a few minutes or working over one hour after which she got extremely exhausted. This patient used to receive over 100 units of long acting insulin (mixtard) per day with a blood sugar level over 300 mg/dl rising frequently to deleterious levels requiring hospitalization. In short her life was miserable and she was severely depressed. With the use of the drug, this patient showed marked improvement within 2-3 wks. She could work for long hours and she was so happy about it that she walked to collage and back daily (over 5 kilometers) her insulin requirements dropped to around 25-30 u/day and her blood sugar dropped to acceptable levels (rarely exceeding 200 mg/dl. Case 8 & 16 showed an equivalent response.

b—Case 8:

A 45 yrs old male that was referred to in the previous section had a near frozen shoulder and was diagnosed as an advanced case of supra spinatous syndrome and was subjected to manipulation under anesthesia which failed to improve his condition within 3 wks of the use of the drug, this patient could move his shoulder painlessly in a more or less normal way.

c—Case 10:

This 40 years old male was emaciated and presented with multiple boils and carbuncles which required frequent surgical interference and that was incapacitating and practically put him out of work for long periods. all this disappeared after treatment with the drug for a few weeks.

e—Case 4:

This 60 years old patient suffered from complete loss of sensation in both soles and could not even feel his own shoes. This symptom lasted 3 yrs. In less than 4 wks of treatment he recovered a good deal of his sensation.

### Mode of Application:

This drug is used for treatment of complications of diabetes mellitus.

### Materials Used for Preparation of the Drug:

1—Licorice root.
2—HCl (4 molar).
3—Sodium carbonate

### Preparation of the Drug in the Form of Syrup:

1—250 gram of licorice root & 0.2 gram of sodium carbonate are added to 500 c.c. of warm water. the mixture is left for 6 hours.
2—750 ml of warm water is added to the mixture and left for 4 hours.
3—Diluted HCl is added then the mixture is stirred and filtered.
4—The filtrate is used as a drug. The dose is 5 ml (one tea spoonful) TDS.

### Preparation of the Drug in the Form of Capsules:

a—The drug is prepared in the form of soft gelatinous capsules according to the following steps:
   1—The solution is prepared according to the above mentioned steps.
   2—Reduction of water content is done by rotary suction pump.
   3—The produced dough is packed in soft gelatinous capsules. each capsule contains a dose equivalent to 5 ml of syrup form.
b—The drug is prepared in the form of hard capsules as follows:
   1—The solution is prepared according to the above mentioned steps.
   2—Dryness is done by lyophilization.
   3—Each capsule contains a dose equivalent to 5 ml syrup form

1- Method of preparing a drug or medication for treatment of diabetes mellitus complications comprising the steps of:
Adding approximately 250 grams of licorice root and approximately 0.2 gram of sodium carbonate to approximately 500 c.c. of warm water to form a first mixture;
Subsequently adding approximately 750 ml of warm water to the first mixture to form a second mixture;
Adding diluted HCl to the mixture followed by stirring the mixture and then filtering the mixture.

2- Method of preparing a drug for treatment of diabetes mellitus complications according to claim 1 characterized in that the first mixture of licorice, sodium carbonate and water is kept for a period ranging from 2-8 hours.

3- Method of preparing a drug for treatment of diabetes mellitus complications according to claim 2 characterized in that the first mixture is kept for a period from 4 to 6 hours.

4- Method of preparing a drug for treatment of diabetes mellitus complications according to claim 2 characterized in that the first mixture is kept for a period of about 6 hours.

5- Method of preparing a drug for treatment of diabetes mellitus complications according to claim 1 characterized in that the second mixture is kept for a period ranging from 2-6 hours.

6- Method of preparing a drug for treatment of diabetes mellitus complications according to claim 5 characterized in that the second mixture is kept for a period of from 3 to 5 hours.

7- Method of preparing a drug for treatment of diabetes mellitus complications according to claim 5 characterized in that the second mixture is kept for a period of about 4 hours.

8- The method of claim 1 including the steps of preparing the medication as a pharmaceutical composition for wherein the drug is concentrated by a rotary suction pump.

9- The method of claim 1 including the steps of preparing the medication as a pharmaceutical composition for wherein the drug is lyophilized and packed in soft gelatinous capsules or hard capsules.

10- A medication comprising a formulation of licorice root, sodium carbonate, HCl and water which is fermented for a period ranging from 6 to 16 hours.

11- The medication of claim 10 being fermented for a period of from 8 to 14 hours.

12- The medication of claim 10 being fermented for a period of about 12 hours.

13- Use of the fermentation product according to claim 10 as a Medicament for treatment of complications of diabetes mellitus.

14- Method for treatment of complications of diabetes mellitus comprises administering an effective amount from drug according to claim 10.

15- Method according to claim 14 wherein the effective dose of the drug ranges from 12 ml to 25 ml.

16- The method according to claim 15 wherein the effective dose is from 15 ml to 20 ml divided into multiple doses.
